(19) United States
(12) Patent Application Publication
(10) Pub. No.: US 2017/0176443 A1
(43) Pub. Date: Jun. 22, 2017

(54) METHOD FOR DETECTING OR MONITORING PROSTATE CANCER

(71) Applicants: Hon Sing Leong, London (CA); Colleen Nicole Biggs, London (CA)

(72) Inventors: Hon Sing Leong, London (CA); Colleen Nicole Biggs, London (CA)

(73) Assignees: ONCOSENSE INC., LONDON ONTARIO (CA); ONCOSENSE INC. (USA), RICHMOND, VT (US)

(21) Appl. No.: 15/451,061

(22) Filed: Mar. 6, 2017

Related U.S. Application Data
(63) Continuation of application No. 14/395,459, filed on Oct. 17, 2014, now abandoned.
(60) Provisional application No. 61/635,692, filed on Apr. 19, 2012.
(60) Provisional application No. 61/791,035, filed on Mar. 15, 2013.

(51) Int. Cl. G01N 33/574

(52) U.S. Cl. CPC G01N 33/57434; G01N 2800/52

(57) ABSTRACT

The present invention provides methods identifying subjects having prostate cancer (PCa) by detecting in microparticles a pair of biomarkers. The methods disclosed can be used to distinguish subjects having PCa from those having non-malignant prostate pathologies, including benign prostatic hyperplasia. Methods for monitoring prostate cancer and assessing efficacy of prostate cancer therapies are also disclosed. Kits for detecting prostate cancer using the methods disclosed are also provided.

### CROSS REFERENCE TO PRIOR APPLICATIONS

This application is a Continuation of U.S. application Ser. No. 14/395,459 filed Oct. 17, 2014, which is a 371 of International Application No. PCT/CA2013/050303 filed Apr. 19, 2013, which claims priority from U.S. Application No. 61/635,692, filed on Apr. 19, 2012 and U.S. Application No. 61/791,035, filed on Mar. 15, 2013, the entire contents of which are incorporated herein by reference.

### FIELD OF THE INVENTION

This invention relates generally to biochemical assays in the field of medicine. In particular, this invention is directed to methods and related materials for detecting and monitoring the progression of cancer, in particular prostate cancer, in human subjects.

### BACKGROUND OF THE INVENTION

Prostate cancer (PCa) is a global health concern. It accounted for 10% of all cancer-related deaths in North America in 2010 (Jemal et al., Cancer Statistics 60:277-300, 2010). The number of men afflicted with PCa is increasing rapidly as the population of males over the age of 50 grows. Thus, strategies for detecting PCa in its early stages are urgently needed.

Conventional PCa screening involves assessing familial history of the disease and screening methods including digital rectal examination (DRE), transrectal ultrasound, and prostate specific antigen (PSA) testing. A subject's physician then uses assessment and screening data to determine whether a prostate biopsy is recommended. Unfortunately, current PCa screening methods result in a high rate of false positives. Large multicenter clinical trials using strict biopsy criteria (i.e., abnormal DRE results and/or a PSA>4 ng/ml) have found a negative biopsy rate of approximately 70% (Thompson et al., New Eng. J. Med. 349:215-224, 2003 and Andriole et al., New Eng. J. Med. 362:1192-1202, 2010).

Biopsies are costly procedures that cause patients pain and anxiety and present a risk to patient health. For example, transrectal guided biopsies cause side effects ranging from temporary erectile dysfunction and blood in the urine, stool and ejaculate, to life-threatening sepsis in a minority of patients (Challacombe et al., BJU Intl. 108:1233-1234, 2011 and Zaytoun et al., Urology 77:910-914, 2011). Means to avoid unnecessary biopsies would benefit patient health and reduce health care costs.

Blood-based tests are advantageous for several reasons, including low invasive sample collection (standard blood draw), low cost and amenability to high throughput analyses. However, the standard blood test for PCa, namely PSA measurement, has a high false positive rate and low specificity. Although PSA is a prostate-specific marker, it is not a PCa-specific marker. Non-malignant conditions, particularly benign prostatic hyperplasia (BPH), can elevate PSA levels in patient serum. BPH is an enlargement of the prostate which can interfere with the normal flow of urine. PSA levels can be elevated in BPH patients due to increased organ volume and inflammation due to associated urinary tract infections. However, BPH is not known to increase a subject's risk of cancer. Because PSA screening does not differentiate between PCa and BPH, even well-controlled studies cite an AUC of approximately 0.6 for detection of PCa based on PSA testing (Aubin et al., J. Urology 184:1947-1952, 2010).

Prostate cancer antigen 3 (PCA3, also referred to as DD3) is specific to human prostate tissue and is overexpressed in prostate cancer (Bussemakers et al., Cancer Res. 59:5975-5979, 1999). Urine tests for PCA3 have lower sensitivity but higher specificity relative to serum PSA tests and a better positive and negative predictive value than PSA (Vlaeminck-Guillem et al., Prog. Urol. 18:259-265, 2008). However, gathering the required urine sample for a PCA3 test is invasive relative to a blood draw. A PCA3 test requires collection of the first portion of urine produced following prostate massage with DRE.

C35 is a protein encoded by C17orf37, which is up-regulated in prostate, breast, ovarian, liver and hepatocellular cancers and colorectal metastases. Advantageously, C35 exhibits a relative lack of expression in healthy tissues. However, currently there are no prostate cancer screening tests that target C35 (Evans et al., Mol. Cancer Ther. 5:291902930, 2006; Dasgupta et al., Oncogene 13:2860-2872, 2009; Wong et al., AACR 101^{st} Annual Meeting 2010; Kilari et al., J. Clin. Oncol. 31:suppl 6; abstract 212, 2013).

A marker that distinguishes between PCa and BPH would be advantageous for PCa screening methods. Such markers have been identified. For example, Ghrelin is a 28 amino acid peptide that is a natural growth hormone secretagogue (GHS) (GS(octanoyl)FLSPEHRQVQQRKESK (SEQ ID NO:1). Ghrelin is known to be co-expressed with its receptor GHSR in human PCa cells. An imaging probe, fluorescein-ghrelin(1-18), that targets receptors for ghrelin can delineate PCa cells from prostate cells having benign disease, including BPH (Lu et al., Prostate 72:825-833, 2012).

Unfortunately, using currently known methodologies, screening for Ghrelin or C35 positive prostate cells would require a biopsy sample from prostate tissue.

Another method that has been used for identifying markers in serum is to test for circulating microvesicles derived from tumor cells. Microvesicles are a type of microparticle (MP), 100 nm-1 μm in diameter, which directly bud from the plasma membrane (Morel et al., Curr. Opin. Hematol. 11:156-164, 2004; Cocucci et al., Trends Cell Biol. 19:43-51, 2009). Microparticles are released by different cells, including tumour cells (Théry et al., Nat. Rev. Immunol. 9:581-593, 2009). The emission of microvesicles, such as exosomes and MPs, is suggested to be involved with tumor progression and metastasis (Schorey, J. Cell. Sci, 123:1603-1611, 2010).

A bead-based method of detecting prostate cancer microvesicles is provided by Caris® Life Sciences, wherein fluorescent beads bound to antibodies to PSMA, PSCA and B7-H3 are used to capture PCa microvesicles. (Kiebel et al., poster, American Urological Association, 2011). However, the bead-based assay does not enumerate PCa microparticles, but rather provides a measurement of fluorescent intensity of the entire sample analyzed. Enumeration of PCa microparticles is desirable, at least because it would provide an indicator of tumor load by relying on the actual number of antigen-positive MPs rather than relative fluorescence of the sample, which may include the binding of soluble protein complexes specific for the antibodies used in the assay.

Use of MPs to detect disease in a subject is further complicated by the fact that the presence of an antigen on a MP does not necessarily classify the cell of origin of the MP in question. For example, in blood, soluble antigens derived from one cell type may adhere to MPs derived from another cell type. Moreover, MPs derived from one cell type may fuse with the membrane of different cell types, which subsequently release MPs (Simak and Gelderman, Transfusion Med. Rev. 20:1-26, 2006). Thus, definitively identifying the origin of circulating MPs has proven challenging.

There is a need in the art to develop a method of detecting PCa that has high specificity and sensitivity. There is also a need for PCa detection method having the capacity to distinguish between PCa and BPH. PCa detection methods having the capacity for enumerative analysis are also desirable.

### SUMMARY OF THE INVENTION

The present invention is broadly summarized as relating to biomarkers suitable for identifying subjects having prostate cancer (PCa). In particular, the biomarkers are present on the surface of prostate cancer microparticles. In one aspect, the invention provides a method for distinguishing patients having PCa from those having non-malignant prostate pathologies, including benign prostatic hyperplasia (BPH), wherein the method comprises identifying the above-mentioned biomarkers.

In a first aspect, the present invention provides a method for detecting prostate cancer in a sample obtained from a subject.

In some embodiments of the first aspect, the method comprises analyzing a bodily fluid sample to detect microparticles having at least first and second biomarkers on their surface in the bodily fluid sample. In some embodiments, the first biomarker is expressed by prostate epithelial cells and the second biomarker is expressed by prostate cancer cells but not by benign prostatic hyperplasia or other non-malignant prostate cells.

In some embodiments of the first aspect, the method comprises comparing the amount of microparticles positive for both the first and second biomarkers with a reference value. In some embodiments, the reference value is derived from a non-malignant prostatic sample. In such embodiments, a detected value above the reference value is indicative of prostate cancer and a detected value equal to or below the reference value is indicative of a non-malignant disease state.

In some embodiments, the reference value is derived from a malignant prostatic sample. In such embodiments, a detected value equal to or above the reference value is indicative of prostate cancer and a detected value below the reference value is indicative of a non-malignant disease state.

In some embodiments of the first aspect, the method comprises diagnosing the subject on the basis of the results obtained in the comparing step.

In some embodiments of the first aspect, the bodily fluid is blood.

In some embodiments of the first aspect, at least two biomarkers are used. In one embodiment, a first biomarker is prostate specific membrane antigen (PSMA), prostate stem cell antigen (PSCA), STEAP1 or STEAP2. In preferred embodiments, the first biomarker is PSMA.

In some embodiments of the first aspect, a second biomarker is Ghrelin or C35. In preferred embodiments, the second biomarker is Ghrelin.

In some embodiments of the first aspect, the method further comprises effectuating a treatment based on the diagnosis determined.

In some embodiments of the first aspect, the analysis of bodily fluid is conducted using a flow cytometry assay. In some embodiments, the flow cytometry assay is fluorescence activated cell sorting (FACS). In some embodiments, the flow cytometry assay is carried out using a nanoscale flow cytometer. In some embodiments, the flow cytometry assay comprises exposing the bodily fluid sample to a composition. The composition in the assay comprising a first labeled binding probe that is specific to the first biomarker and a second labeled binding probe that is specific to the second biomarker. In some embodiments, the labels of the first and second probes are distinguishable. In preferred embodiments, the first labeled binding probe is anti-PSMA-RPE IgG and the second labeled probe is Ghrelin-Cy5 or Ghrelin-FITC.

In some embodiments of the first aspect, analysis is carried out with reference to negative controls of the first and second binding probes using first and second negative control binding probes. In preferred embodiments, the first negative control binding probe is mouse IgG-RPE and the second negative control binding probe is des-acyl Ghrelin-Cy5 or des-acyl Ghrelin-FITC.

In some embodiments of the first aspect, the reference value represents the amount of microparticles positive for the first and second biomarkers in a sample obtained from a subject having a non-malignant prostate or benign prostatic hyperplasia (BPH) and the difference is an increase. In preferred embodiments, the reference value is in a range of 14,000 to 22,000 PCMP counts/μL.

In a second aspect, the present invention provides a diagnostic assay for prostate cancer.

In some embodiments of the second aspect, the diagnostic assay comprises analyzing a bodily fluid sample to detect microparticles having first and second biomarkers on their surface in the bodily fluid sample, wherein the first biomarker is expressed by prostate epithelial cells and the second biomarker is expressed by prostate cancer (PCa) cells but not by benign prostatic hyperplasia or other non-malignant prostate cells; comparing the amount of microparticles positive for both the first and second biomarkers with a reference value, wherein if the reference value is derived from a non-malignant prostatic sample then a detected value above the reference value is indicative of prostate cancer and a detected value equal to or below the reference value is indicative of a non-malignant disease state and, wherein if the reference value is derived from a malignant prostatic sample then a detected value equal to or above the reference value is indicative of prostate cancer and a detected value below the reference value is indicative of a non-malignant disease state; and diagnosing the subject on the basis of the results obtained the comparison step.

In a third aspect, the present invention provides a method for monitoring prostate cancer in a subject.

In some embodiments of the third aspect, the method for monitoring prostate cancer comprises analyzing a first bodily fluid sample, wherein the first sample was obtained from the subject at a first time point, to detect microparticles having at least first and second biomarkers on their surface in the bodily fluid sample. In some embodiments, the first biomarker is expressed by prostate epithelial cells and the second biomarker is expressed by prostate cancer (PCa) cells but not by benign prostatic hyperplasia or other non-malignant prostate cells.

In some embodiments of the third aspect, the method for monitoring prostate cancer comprises comparing the amount of microparticles positive for both the first and second biomarkers with a reference value, wherein if the reference value is derived from a non-malignant prostatic sample then a detected value above the reference value is indicative of prostate cancer and a detected value equal to or below the reference value is indicative of a non-malignant disease state and, wherein if the reference value is derived from a malignant prostatic sample then a detected value equal to or above the reference value is indicative of prostate cancer and a detected value below the reference value is indicative of a non-malignant disease state.

In some embodiments of the third aspect, the method for monitoring prostate cancer comprises diagnosing the subject on the basis of the results obtained in the comparing step.

In some embodiments of the third aspect, the method for monitoring prostate cancer comprises effectuating a treatment regimen based diagnosis obtained in the diagnosing step.

In some embodiments of the third aspect, the method for monitoring prostate cancer comprises analyzing a second bodily fluid sample, wherein the second sample was obtained from the subject at a second time point, to detect microparticles having at least first and second biomarkers on their surface in the bodily fluid sample, wherein the first biomarker is expressed by prostate epithelial cells and the second biomarker is expressed by prostate cancer (PCa) cells but not by benign prostatic hyperplasia or other non-malignant prostate cells.

In some embodiments of the third aspect, the method for monitoring prostate cancer comprises comparing the amount of microparticles positive for both the first and second biomarkers with a reference value, wherein if the reference value is derived from a non-malignant prostatic sample then a detected value above the reference value is indicative of prostate cancer and a detected value equal to or below the reference value is indicative of a non-malignant disease state and, wherein if the reference value is derived from a malignant prostatic sample then a detected value equal to or above the reference value is indicative of prostate cancer and a detected value below the reference value is indicative of a non-malignant disease state.

In some embodiments of the third aspect, the method for monitoring prostate cancer comprises comparing the amount of microparticles positive for the first and second biomarker in the second bodily fluid sample with a the value obtained in first comparing step wherein an increase in the amount of microparticles positive for the first and second biomarkers in the second sample relative to the value obtained in the first comparing step is indicative of a worsened disease state and a decrease in the amount of microparticles positive for the first and second biomarkers in the second sample relative to the value obtained in the first comparing step is indicative of an improved disease state.

In some embodiments of the third aspect, the method for monitoring prostate cancer comprises diagnosing any change in the subject's disease state on the basis of the results obtained by comparing the amount of dual positive microparticles in the first and second samples.

In a fourth aspect, the present invention provides a method for assessing efficacy of a therapy on a subject having prostate cancer.

In some embodiments of the fourth aspect, the method for assessing efficacy of a therapy on a subject having prostate cancer comprises: analyzing a bodily fluid sample from a subject, wherein the subject has be subjected to a prostate cancer therapy, to detect microparticles having at least first and second biomarkers on their surface in the bodily fluid sample, wherein the first biomarker is expressed by prostate epithelial cells and the second biomarker is expressed by prostate cancer (PCa) cells but not by benign prostatic hyperplasia or other non-malignant prostate cells.

In some embodiments of the fourth aspect, the method for assessing efficacy of a therapy on a subject having prostate cancer comprises comparing the amount of microparticles positive for both the first and second biomarkers with a reference value, wherein if the reference value is derived from a non-malignant prostatic sample then a detected value above the reference value is indicative of prostate cancer and a detected value equal to or below the reference value is indicative of a non-malignant disease state and, wherein if the reference value is derived from a malignant prostatic sample then a detected value equal to or above the reference value is indicative of prostate cancer and a detected value below the reference value is indicative of a non-malignant disease state; and

In some embodiments of the fourth aspect, the method for assessing efficacy of a therapy on a subject having prostate cancer comprises diagnosing the efficacy of the therapy as good if the value obtained in the comparing step indicates a non-malignant disease state or poor if the value obtained in the comparing step indicates prostate cancer.

In a fifth aspect, the present invention provides a kit for detecting prostate cancer in a bodily fluid sample.

In some embodiments of the fifth aspect, the kit comprises a first binding probe specific to a biomarker that is expressed by prostate epithelial cells and a second binding probe specific to a biomarker that is expressed by prostate cancer (PCa) cells but not by benign prostatic hyperplasia or other non-malignant prostate cells.

In some embodiments of the fifth aspect, the first binding probe anti-PSMA-RPE IgG. In some embodiments, the second binding probe is Ghrelin-Cy5 or Ghrelin-FITC.

In some embodiments of the fifth aspect, the kit comprises a first negative control binding probe specific to mouse IgG. In some embodiments, the kit comprises a second negative control binding probe specific to des-acyl Ghrelin.

In some embodiments of the fifth aspect, the first negative control binding probe is the monoclonal antibody mouse IgG-RPE. In some embodiments, the second negative control binding probe is des-acyl Ghrelin-Cy5 or des-acyl Ghrelin-FITC. In preferred embodiments of the fifth aspect, the kit comprises a first and second sealed container, wherein the first sealed container comprises anti-PSMA-RPE IgG and Ghrelin-Cy5 or Ghrelin-FITC and the second sealed container comprises mouse IgG-RPE and des-acyl Ghrelin-Cy5 or des-acyl Ghrelin-FITC.

In some embodiments of the fifth aspect, the kit comprises a carrier, wherein a carrier is a box, carton, or tube. In some embodiments, the carrier comprises one or more sealed containers, wherein the one or more sealed container is a vial, tube, ampoule, bottle, pouch or envelope.

In some embodiments of the fifth aspect, the kit comprises one or more media, media ingredients or reagents for measurement of at least one of the first and second biomarkers. In some embodiments, the one or more reagents are buffers or probes.

In some embodiments of the fifth aspect, the kit comprises one or more instructions or protocols for carrying out the methods of the present invention.

### BRIEF DESCRIPTION OF THE DRAWINGS

Features of the invention will become more apparent in the following detailed description in which reference is made to the appended drawings wherein:

FIG. 1. depicts nanoscale flow cytometry of prostate cancer microparticles in plasma from healthy volunteers. The top panel reveals the size distribution of dual-positive microparticles (events that bind anti-PSMA-RPE IgG and Ghrelin-Cy5 peptide) present in the red gate of the bottom histoplot in this representative patient plasma sample. Events in the red gate represent dual-positive events that are not present in the isotype stained control of the same but separately stained plasma sample.

FIG. 2. depicts nanoscale flow cytometry of prostate cancer microparticles in plasma from patients with benign prostatic hyperplasia. The top panel reveals the size distribution of dual-positive microparticles (events that bind anti-PSMA-RPE IgG and Ghrelin-Cy5 peptide) present in the red gate of the bottom histoplot in this representative patient plasma sample. Events in the red gate represent dual-positive events that are not present in the isotype stained control of the same but separately stained plasma sample.

FIG. 3. depicts nanoscale flow cytometry of prostate cancer microparticles in plasma from patients with localized prostate cancer. The top panel reveals the size distribution of dual-positive microparticles (events that bind anti-PSMA-RPE IgG and Ghrelin-Cy5 peptide) present in the red gate of the bottom histoplot in this representative patient plasma sample. Events in the red gate represent dual-positive events that are not present in the isotype stained control of the same but separately stained plasma sample.

FIG. 4. depicts nanoscale flow cytometry of prostate cancer microparticles in plasma from patients with metastastic prostate cancer. The top panel reveals the size distribution of dual-positive microparticles (events that bind anti-PSMA-RPE IgG and Ghrelin-Cy5 peptide) present in the red gate of the bottom histoplot in this representative patient plasma sample. Events in the red gate represent dual-positive events that are not present in the isotype stained control of the same but separately stained plasma sample.

FIG. 5. is a graphic representation showing counts of prostate cancer microparticles (PCMPs) in patients with BPH and patients with PCa. PCMPs are defined as dual-positive for anti-PSMA-RPE IgG and Ghrelin-Cy5 peptide. All patients had PSA>4 ng/mL.

FIG. 6. is a graphic representation showing counts of prostate microparticles (PSMA+ve only) in patients with BPH and patients with PCa. Prostate microparticles are defined as sub-micron events that bind only the anti-PSMA-RPE IgG. PSA>4 ng/mL for all patient plasmas and N>20 each group.

FIG. 7. depicts monitoring of changes in patient PCMP levels before and after prostatectomy. For each sample ID (HL XXX), fold difference is recited in the right column. Values in red (marked by an up arrow) indicate an increase in PCMP concentration after prostatectomy. Values in black (without arrows) indicate a decrease in PCMP concentration after prostatectomy.

### DETAILED DESCRIPTION OF THE INVENTION

The definitions of certain terms as used in this specification are provided below. Unless defined otherwise, all technical and scientific terms used herein generally have the same meaning as commonly understood by one of ordinary skill in the art to which this invention belongs.

As used herein, the term “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

As used herein, the terms “diagnose”, “diagnosing” and “diagnostic” refer to the process of determining a disease state or disorder in a subject. In determining disease state a diagnostician might classify one or more characteristics of a subject, such as, for example, symptoms and/or biomarkers. A “diagnostic assay” is referred to herein as a tool that a diagnostician might use to narrow the diagnostic possibilities.

As used herein, the term “subject” refers to a mammal, such as, for example, a human, non-human primate, mouse, rat, dog, cat, horse, or cow. In some embodiments, a subject is human and might be referred to as a patient. A subject can be one who has been previously diagnosed or identified as having a disease, and optionally one who has already undergone, or is undergoing, a therapeutic intervention for a disease. Alternatively, a subject can also be one who has not been previously diagnosed as having a disease.

As used herein, the terms “prostate cancer” and “prostate malignancy” refers to a prostate containing tumor-forming prostate epithelial cells. Conversely, a “non-malignant prostate”, as used herein, refers to a prostate that does not contain tumor-forming prostate epithelial cells.

As used herein, the term benign prostatic hyperplasia or “BPH” refers to an increase in size of a prostate due to an increase in the number of prostate cells. BPH is not known to cause cancer, including prostate cancer, or to increase the risk of cancer, including prostate cancer.

As used herein, the terms “bodily fluid sample” and “fluid sample” refer to a specimen obtained from a subject. In some embodiments, the sample comprises blood, a fraction of blood or urine.

As used herein, the terms “detect”, “detection” and “detecting” refer to a quantitative or qualitative determination of a property of an entity, for example, quantifying the amount or concentration of a molecule or the activity level of a molecule. The term “concentration” or “level” can refer to an absolute or relative quantity. Measuring a molecule may also include determining the absence or presence of the molecule. Various methods of detection are known in the art, for example fluorescence analysis. In this regard, biomarkers can be measured using fluorescence detection methods or other methods known to the skilled artisan.

As used herein, the terms “microparticle” or “MP” refer to small membrane bound vesicles (i.e., generally 100 nm to 1 μm in diameter) that directly bud from the plasma membrane of various cells, including tumor cells, or are storage vesicles released by prostate cells or prostate cancer cells by exocytosis. Microparticles circulate in blood that is derived from cells in contact with the bloodstream, such as, for example, endothelial cells. Microparticles are useful in various embodiments of the present invention, at least because they retain at least some of the membrane protein characteristics of their parent cells.

As used herein, the term “biomarker” refers to a molecule whose measurement provides information regarding the state of a subject, or a feature of a subject, such as, for example, an organ, tissue, system or cell. For example, the disease state of a subject can be assessed using a biomarker. Measurements of a biomarker may be used alone or combined with other data obtained regarding a subject, or feature thereof, in order to determine the state of the subject, or feature thereof. In one embodiment, the biomarker is “differentially present” in a sample taken from a subject of one disease state (e.g., having a disease) as compared with another disease state (e.g., not having the disease). In one embodiment, the biomarker is “differentially present” in a sample taken from a subject undergoing no therapy or one type of therapy as compared with another type of therapy. Alternatively, the biomarker may be “differentially present” even if there is no known difference in disease state, e.g., the biomarkers may allow the detection of asymptomatic risk.

As used herein, the terms “specific” and “specificity” refer to the nature of the binding of a biomarker with its binding probe. “Specific binding” or “selective binding” refers to a probe that binds a biomarker with a specificity sufficient to differentiate between the biomarker and other components or contaminants of a test sample.

As used herein, the term “reference value” refers to a baseline value. In some embodiments, a baseline value can represent the amount of MPs in a composite sample from an effective number of subjects who do not have the disease of interest but are positive for both of the biomarkers of interest. In some embodiments, a reference value can also comprise the amount of MPs in a composite sample from an effective number of subjects who have the disease of interest, as confirmed by an invasive or non-invasive technique.

As used herein, the terms “indicative of”, “associated with” and “correlated to” refer to the determination of a relationship between one type of data with another or with a state. In some embodiments, correlating the measurement with disease comprises comparing the amount of MPs positive for a pair of biomarkers with a reference value. In some embodiments, correlating the measurement with disease comprises determining the subject's disease state.

As used herein, the terms “treatment”, “treatment regimen”, “therapy” and “therapeutic treatment” refer to an attempted remediation of a health problem. In some embodiments, treatment can be selected from, administering a disease-modulating drug to a subject, administering disease-modulating radiation to a subject, surgery or scheduling a further appointment with a medical practitioner. Treatment refers to one or more of initiating therapy, continuing therapy, modifying therapy or ending therapy.

As used herein, the terms “prophylaxis” and prophylactic” refer to measures taken to prevent disease. Prophylactic treatment includes, for example, measures to reverse, prevent or slow physiological features that are precursors to disease.

As used herein, the terms “binding probe” or “binding ligand” refer to compounds that are used to detect the presence of, or to quantify, relatively or absolutely, a target molecule or target sequence and that will bind to the target molecule or sequence, either directly or indirectly. Generally, a binding probe allows attachment of a target molecule or sequence to the probe for the purpose of detection. In some embodiments, the target molecule or sequence is a biomarker. It follows that the composition of the binding probe will depend on the composition of the biomarker. Binding probes for a variety of biomarkers are known or can be generated using known techniques. For example, when the biomarker is a protein, the binding probes include proteins, such as, for example, antibodies or fragments thereof or small molecules.

As used herein, the terms “label” and “labeled” refer to a composition detectable by spectroscopic, photochemical, biochemical, immunochemical, chemical, or other physical means. A compound that is labeled has at least one molecule, element, isotope or chemical compound attached to it to enable the detection of the compound. For example, useful labels include fluorescent dyes, which might also be referred to as fluorophores.

As used herein, the term “fluorophore” refers to a molecule or part of a molecule that absorbs energy at one wavelength and re-emits energy at another wavelength. Detectable properties of fluorophores include fluorescence intensity, fluorescence lifetime, emission spectrum characteristics, energy transfer, and the like. Fluorophores are of use in the present invention, at least due to their strong signals, which provide a signal-to-noise ratio sufficient to allow interpretation of the signals. Suitable fluorophore for use in the present invention include, but are not limited to, fluorescent lanthanide complexes, including those of Europium and Terbium, fluorescein, rhodamine, tetramethylrhodamine, eosin, erythrosin, coumarin, methyl-coumarins, pyrene, Malacite green, stilbene, Lucifer Yellow, Cascade Blue, Texas Red, Alexa dyes and others described in the 6th Edition of the Molecular Probes Handbook by Richard P. Haugland.

As used herein, the terms “nanoscale flow cytometer” or “nanoscale flow cytometry” refer to a flow cytometry device, or a process of using said flow cytometry device, that can analyze events that are 1000 nm-100 nm in diameter.

As used herein, the term “antibody” refers to a protein comprising one or more polypeptides substantially encoded by all or part of immunoglobulin genes known to the skilled artisan. The immunoglobulin genes recognized by a skilled artisan include, for example in humans, the kappa, lambda and heavy chain genetic loci, which together compose myriad variable region genes, and constant region genes mu, delta, gamma, epsilon and alpha, which encode IgM, IgD, IgG, IgE, and IgA isotypes respectively. Antibody herein is meant to include full length antibodies and antibody fragments, and may refer to a natural antibody from any organism, an engineered antibody or an antibody generated recombinantly for experimental, therapeutic or other purposes as further defined below. The term “antibody” refers to both monoclonal and polyclonal antibodies. Antibodies can be antagonists, agonists, neutralizing, inhibitory or stimulatory.

As used herein the term “negative control” refers to an element or group used in an experiment to ensure that a negative result is produced when a negative result is expected. For example, a negative control binding probe, as referred to herein, is a probe that should not bind to the MP being examined, because the probe's target is not present in the sample being examined. Thus, when assayed, if a negative control binding probe successfully binds to a MP, then then it can be inferred that a confounding variable acted on the experiment, suggesting that the positive results are likely not due the intended specific binding.

As used herein, the term “monitoring” refers to observation of a disease over time. Monitoring of a subject's disease state can be performed by continuously measuring certain parameters and/or performing a medical test repeatedly. In some embodiments of the present invention, a subject's disease state is monitored by obtaining bodily fluid samples repeatedly, assaying the samples using the method disclosed herein and comparing assay results with one another and with a reference value to identify any change in the subject's disease state.

As used herein, the term “disease state” refers to any distinguishable manifestation of a particular disease, including non-disease. For example, disease state includes, without limitation, the presence or absence of a disease, the risk of developing a disease, the stage of a disease, the progression or remission of a disease over time and the severity of disease. The term “worsened disease state” refers to the progression of a disease over time. The term “improved disease state” refers to remission of disease over time.

As used herein, the term “efficacy” refers to the capacity of an intervention to produce a therapeutic effect. For example, a PCa treatment having good efficacy might significantly reduce or eliminate from a subject detectable tumor-forming prostate epithelial cells. In contrast, a PCa treatment having a poor efficacy might not reduce in a subject the level of detectable tumor-forming prostate epithelial cells.

As used herein, the term “kit” refers to a collection of elements that together are suitable for a defined use.

As used herein, the term “invasive” refers to a medical procedure in which a part of the body is entered. In some embodiments, entry into the body might cause a subject to feel pain during or following the procedure. For example, surgical procedures involving incisions are invasive. Herein, a standard blood draw is not considered to be invasive.

The present invention generally relates to a non-invasive means of screening a subject for PCa. The invention is based on the inventors' observations that i) MPs found in mammalian plasma can be identified and enumerated using, for example, flow cytometry, ii) prostate cells or prostate cancer cells undergo extravasation, apoptosis or necrosis, releasing prostate MPs into the circulatory system; and iii) prostate MPs can be distinguished as cancerous or non-cancerous by quantifying the MPs positive for a pair of surface biomarkers using flow cytometry. The pair of biomarkers includes a biomarker specific to prostate cells that are not typically found in healthy individuals, such as, for example, prostate-specific membrane antigen (PSMA) and a biomarker that is specific to PCa cells. In some aspects of the invention, the PCa-specific biomarker is not significantly expressed in non-malignant prostate cells, including BPH cells, and is not present on the surface of non-malignant prostate MPs, including BPH MPs. In some aspects of the invention, the PCa-specific biomarker is present at a level below a reference value in prostate and BPH MPs.

Some embodiments of the present invention involve a method for diagnosing PCa in a subject. In some embodiments, the method comprises obtaining a bodily fluid sample from the subject, preferably a blood sample. In some embodiments of the method, the blood sample can be fractionated to obtain platelet poor plasma. The sample is then analyzed by, for example, a flow cytometry assay that specifically detects MPs positive for first and second biomarkers in the sample. The first biomarker is expressed in prostate epithelial cells.

The first biomarker is preferably PSMA, which is known to be specifically expressed on the surface of prostate cells and some prostate cancer cells. It is contemplated that PSCA, STEAP1 or STEAP2 could also be used as the first biomarker, at least because PSCA, STEAP1 and STEP2 are known to be expressed in prostate epithelial cells and therefore predicted to be present in prostate microparticles. It follows that the presence of PSMA, PSCA, STEAP1 or STEAP2 would be sufficient to identify MPs of prostatic origin, as exemplified by prostate MPs positive for PSMA. However, the presence of PSMA on the surface of a MP alone cannot identify the MP as being a PCa MP, at least because BPH cells and MPs have a detectable level of PSMA on their surfaces. Further, the inventors are unaware of any evidence to suggest that PSCA, STEAP1 or STEAP2 would be useful for distinguishing PCa cells from non-malignant prostate cells.

The second biomarker is expressed by PCa cells but is not significantly expressed by BPH or other non-malignant prostate cells. As indicated above, PSMA is not sufficient to distinguish between subjects having PCa and BPH. A subpopulation of subjects having BPH has MPs that are PSMA positive. Another sub-population of subjects having BPH has a low amount of MPs that are PSMA positive. Such low levels of PSMA are below the reference value disclosed herein.

In some embodiments of the present invention, the second biomarker is Ghrelin. Ghrelin is a hunger-stimulating peptide and hormone that has a G protein-coupled receptor called the growth hormone secretagogue receptor. Ghrelin is expressed on the surface of a variety of cells. However, Ghrelin is not significantly expressed in non-malignant prostate cells, including BPH cells, nor is it present on the surface of non-malignant prostate MPs, including BPH MPs.

Detection of MPs positive for both PSMA and Ghrelin allows for specific identification of samples originating from subjects having PCa.

It is contemplated herein that the second biomarker could be C35. C35 is specific to cancer cells, including prostate cancer cells and not expressed in corresponding healthy cells. Thus, detection of MPs positive for C35 would be indicative of cancer. It follows that detection of MPs positive for Ghrelin or C35 and at least one of PSMA, PSCA, STEAP1 or STEAP2 would allow for specific identification of samples originating from subjects having PCa.

In some embodiments of the present invention, the amount of MPs having both the first and second biomarkers on their surface is compared with a reference value.

The reference value can be a baseline amount that represents the amount of microparticles having both the first and second biomarkers on their surface that are found in a given volume of sample from a subject who do not have the disease of interest. Where a reference value is indicative of a subject having a non-malignant prostate, a value greater than said reference value would be indicative of prostate cancer. It is also contemplated herein that a reference value could, in contrast, represent the amount of microparticles positive for both first and second biomarkers that are found in a given volume of sample from a subject having the disease of interest. Where a reference value is indicative of a subject having prostate cancer, a value less than said reference value would be indicative of a non-malignant prostate. In some embodiments of the present invention, the reference value is in a range of 14,000 to 22,000 PCMP counts/μL and a value above 14,000 to 22,000 PCMP counts/μL is indicative of prostate cancer.

In some embodiments of the present invention, the method can yield a result indicative of prostate cancer. Treatments for prostate cancer are known in the art. A treatment for prostate cancer can be selected from, for example, administering a chemotherapeutic agent to a subject, administering disease-modulating radiation to a subject, surgery or scheduling a further appointment with a medical practitioner.

In some embodiments of the present invention, the method can yield a result indicating that prostate cancer is not present in the patient sample. In such instance, further monitoring of the patient may be recommended by way of further tests or visits to a medical practitioner over time.

In some embodiments of the present invention, the preferred flow cytometry assay comprises exposing the sample to a composition, the composition comprising a first labeled binding probe that is specific to the first biomarker and a second labeled binding probe that is specific to the second biomarker. It is contemplated that flow cytometry instruments known to the skilled artisan are suitable for use with the present invention, at least for example, instruments suitable for standard flow cytometry, nanoscale flow cytometry or FACS. In some embodiments, the first and second binding probes are labeled with fluorophores. When selecting suitable fluorophores it is important that the excitation wavelength of the fluorophore conjugated to the first binding probe is distinct from the excitation wavelength of the fluorophore conjugated to the second binding probe.

Suitable fluorophores for use in the present invention include, but are not limited to, fluorescent lanthanide complexes, including those of Europium and Terbium, fluorescein, rhodamine, tetramethylrhodamine, eosin, erythrosin, coumarin, methyl-coumarins, pyrene, Malacite green, stilbene, Lucifer Yellow, Cascade Blue, Texas Red, Alexa dyes and others described in the 6th Edition of the Molecular Probes Handbook by Richard P. Haugland. In some embodiments of the present invention R-phycoerythrin (RPE) is conjugated to the first binding probe and flourescein isothiocyanate (FITC) is conjugated to the second binding probe.

In some embodiments of the present invention, negative controls are used in the method of detecting prostate cancer to allow for enumeration of microparticles that are positive for the first and/or second biomarkers.

In some embodiments, the first negative control is mouse IgG-RPE and the second negative control is and des-acyl Ghrelin-Cy5 or des-acyl Ghrelin-FITC.

In some embodiments of the methods of the present invention, a portion of the sample of the bodily fluid is removed from the sample and exposed to a composition comprising binding probes specific to the first and second negative controls. The exposed sample is then analyzed by a flow cytometry assay that specifically detects microparticles having both the first and second biomarkers on their surface in the bodily fluid sample. If any microparticles are found to bind to one or more of the negative control probes, then a confounding variable might be responsible for any fluorescent microparticles that are identified in the disclosed assay for detecting microparticles having both first and second biomarkers on their surface. If the fluorescence of the negative control probes is not observed, then confounding variables can be eliminated as possible cause for positive results that are found in the disclosed assay for detecting microparticles having both first and second biomarkers on their surface.

In some embodiments of the present invention, a diagnostic assay for prostate cancer is provided, wherein the assay comprises the method set forth above, and disclosed further in the examples herein.

In some embodiments of the present invention, the method provides a less-invasive method for detecting prostate cancer in a subject, relative to biopsy methods and PCA3 assays known in the art. Advantageously, some embodiments of the present invention provide a method for detecting prostate cancer that results in fewer false positive than current blood-based PSA tests. Further, in addition to being amenable to high throughput, methods of the present invention involve identifying dual-positive microparticles (e.g., Ghrelin+ and PSMA+). In contrast, many existing technologies such as ELISA and western immunoblotting, cannot address two parameters simultaneously. In some embodiments, the methods of the present invention also provide enumeration of single or dual positive microparticles when negative controls are also analyzed. In some embodiments, enumeration allows a diagnostician to assess the impact of a therapeutic intervention by enumerating the total change in an amount of prostate cancer microparticles before and therapy.

In some embodiments of the present invention, a method for monitoring prostate cancer in a subject is provided. In some monitoring methods of the present invention a first fluid sample is obtained from the subject at a first time point. The first sample is then subjected to analysis and comparison to a reference value, as set forth above and described further in the examples below. A treatment regimen can then be effectuated based on the value obtained from the first sample. The treatment might involve, for example, drug, radiation or surgical intervention or it might involve further monitoring as discussed below.

In some embodiments, the monitoring method of the present invention further comprises, for example, obtaining a second bodily fluid sample from the subject at a second time point. The second sample is then subjected to analysis and comparison to a reference value, as set forth above and described further in the examples below. The reference value obtained in the second sample is then compared to the reference value, to determine if prostate cancer is present, and the value obtained from the first sample to determine if the subject's disease state has improved, worsened or remained constant since the first time point.

In some embodiments, monitoring using the method of the present invention can involve the collecting, analyzing and comparing the analytical results from a series of samples taken from the patient over a series of time periods.

In some embodiments, the monitoring method of the present invention also provides an opportunity to assess the efficacy of one or more treatments that were provided to the subject during the time between samples obtained from the subject. A subsequent reference value indicating improved disease state would be indicative of a treatment having good efficacy. A subsequent reference value indicating worsened disease state would be indicative of a treatment having poor efficacy.

In some embodiments of the present invention, a method is provided for assessing efficacy of a therapy on a subject having prostate cancer, wherein repeated sampling of a patient is not required. In such methods, a bodily fluid sample is obtained from a subject that has been treated with a prostate cancer therapy. The sample is then analyzed and compared to a reference sample as set forth above and described further in the examples below. The reference value obtained in from the sample is then compared to the reference value to determine if prostate cancer is present. Such a method might be advantageous for determining whether surgery, such as, for example, radical prostatectomy, has successfully removed all PCa tissue.

In some embodiments of the present invention, a kit is provided for detecting prostate cancer in a bodily fluid sample. In some embodiments, the kit comprises a first binding probe that is expressed by prostate epithelial cells, such as, for example, PSMA, PSCA, STEAP1 or STEAP2, and a second binding probe specific to a biomarker that is expressed by prostate cancer (PCa) cells but not by BPH or other non-malignant prostate cells. In some embodiments, the biomarker that is expressed by PCa cells but not by BPH or other non-malignant prostate cells is Ghrelin or C35. The first biomarker must also be present on the surface of microparticles derived from parent PCa cells. The second biomarker must also be present on the surface of MPs derived from parent PCa cells and must not be present on the surface of MPs derived from parent BPH or non-malignant prostate cells.

First and second binding probes might be commercially available or they might be prepared by a skilled artisan, at least because the sequence and structure of PSMA, PSCA, STEAP1, STEAP2, Ghrelin and C35 are known in the art.

In some embodiments, the kit comprises anti-PSMA-RPE IgG and Ghrelin-Cy5 or Ghrelin-FITC.

In some embodiments, the kit of the present invention also comprises first and second negative control binding probes. In some embodiments the negative control binding probes are mouse IgG-RPE and des-acyl Ghrelin-Cy5 or des-acyl Ghrelin-FITC.

In some embodiments, the kit of the present invention provides the first and second binding probes in a first sealed container. In some embodiments, the negative controls are provided in a second sealed container.

In some embodiments, the kits of the present invention might comprise a carrier, such as a box, carton, tube or the like, having disposed therein one or more sealed containers, such as vials, tubes, ampoules, bottles, pouches, envelopes and the like. In some embodiments, the kit might comprise one or more media or media ingredients or reagents for measurement of the various biomarkers disclosed herein. For example, kits of the invention may also comprise, in the same or different containers, one or more suitable buffers or probes. The kits of the present invention may also comprise one or more instructions or protocols for carrying out the methods of the present invention.

The invention will be more fully understood upon consideration of the following non-limiting Examples.

### EXAMPLES

The present invention is further illustrated by the following examples, which should not be construed as limiting in any way.

### Example 1: Materials and Methods

Subjects: Patients were recruited under three REB approved ethics applications, REB103156, REB100960, and REB 18632E.

The patient group made up of patients with BPH included males who were 50+ years old, exhibited serum PSA levels greater than 4 ng/mL and whose prostate biopsy yielded no prostate cancer based on pathology reports (N>20).

The patient group made up of patients with localized prostate cancer included males who were 50+ years old, exhibited serum PSA levels greater than 4 ng/mL and whose prostate biopsy yielded evidence of prostate cancer, with a Gleason Score of 6 or above. All patients in this group were candidates for, or had been subjected to, radiation therapy or prostatectomy at the time of blood collection (N>25).

The patient group made up of patients with metastatic prostate cancer included males who were 50+ years old, had received some form of treatment (e.g., radiation therapy or prostatectomy) and who had a relapse of prostate cancer years later, as evidenced by rising levels of PSA, determined as PSA>2 ng/mL. A subpopulation of these patients had positive radiographic bone scans indicating the presence of metastatic PCa bone lesions (N>20).

Patients that were monitored for changes in PCa microparticles after prostatectomy included males who were 50+ years old who had prostate cancer with a Gleason score of >6, had a pre-surgery serum PSA value of 4>ng/mL and had a tumor volume of at least 20 mL (N>20).

Plasma Preparation:

7 ml blood was collected from each subject into Sodium-Heparin BD Vaccutainers (BD Biosciences; Cat#3678800). To separate plasma from erythrocytes, blood was spun down at 1500 gs for 10 minutes at 24° C. in an Eppendorf Centrifuge 5810 R. Plasma was removed from the vaccutainer in 1 mL quantities and transferred into 1.7 mL microtubes tubes (Frogga Bio; Cat#1260-00). To remove residual platelets or erythrocytes microtubes were spun down at 7000 rpm for 5 minutes at room temperature in Eppendorf Centrifuge 5415 C. Plasma was transferred into 1.5 mL cryovials (Sarstedt; Cat#72.694.006) in 0.5 mL aliquots and stored at −80° C.

Antibody Conjugation:

Anti-PSMA antibody that binds to the extracellular domain of PSMA was conjugated to a Phycoerythrin fluorophore using the Lightning-Link R-Phycoerythrin conjugation kit (Innova Biosciences; Cat#703-0010). Antibody was aliquoted and stored at −20° C.

Purified mouse IgG1, κ Isotype Ctrl (Biolegend; Cat#401402) was conjugated to a Phycoerythrin fluorophore using the Lightning-Link R-Phycoerythrin conjugation kit (Innova Biosciences; Cat#703-0010). Antibody was aliquoted and stored at −20° C.

Sample Preparation (using Ghrelin-Cy5 Peptide): The following procedure was performed in the dark to protect light sensitive reagents. 1 μL of Anti-PSMA-RPE antibody (408.42 ug/mL) and 1 μL of LCE 00242-Cy5 (62.5 μM) were added to 20 μL of patient plasma in microtube. The samples were left to incubate in the dark at room temperature for 30 minutes. After incubation, samples were diluted in 600 μL sterile double-distilled Milli-Q water.

The sequence of the Ghrelin-Cy5 binding probe LCE00242 is: H-GS-Dpr(octanoyl)-FLSPEHRQVQQRKES-K(Cy5)-NH2 (SEQ ID NO:2).

Sample Preparation (isotype negative control for Ghrelin-Cy5 Peptide): The following procedure was performed in the dark to protect light sensitive reagents. 1 μL of Mouse IgG-RPE antibody (408.42 μg/mL) and 1 μL of LCE 00254-Cy5 (62.504) were added to 20 μL of patient plasma in microtube. The samples were left to incubate in the dark at room temperature for 30 minutes. After incubation, samples were diluted in 600 μL sterile double-distilled Milli-Q water.

The sequence of the des-acyl Ghrelin-Cy5 binding probe LCE00254 is: H-GSSFLSPEHRQVQQRKES-K(Cy5)-NH2 (SEQ ID NO:3).

Sample Preparation (Using Ghrelin-FITC Peptide): The following procedure was performed in the dark due to light sensitive reagents. 1 μL of Anti-PSMA-PE antibody (408.42 μg/mL) and 1 uL of Ghrelin-FITC (0.125 mM) were added to 20 μL of patient plasma in microtube. The samples were left to incubate in the dark at room temperature for 30 minutes. After incubation, samples were diluted in 600 μL sterile double-distilled Milli-Q water.

The sequence of the Ghrelin-FITC binding probe LCE0080 is: H-GS-Dpr(octanoyl)-FLSPEHRQVQQRKES-K(FITC)-NH2 (SEQ ID NO:4).

Sample Preparation (isotype negative control of Ghrelin-FITC Peptide): 1 μL of Mouse IgG-RPE antibody (408.42 μg/mL) and 1 μL of LCE00203-FITC (0.125 mM) were added to 20 μL of patient plasma in microtube. The samples were left to incubate in the dark at room temperature for 30 minutes. After incubation, samples were diluted in 600 μL sterile double-distilled Milli-Q water.

The sequence of the des-acyl Ghrelin-FITC binding probe LCE00203: H-GSSFLSPEHRQVQQRKES-K(FITC)-NH2 (SEQ ID NO:5).

Sample Analysis:

Samples were analyzed using the Apogee A50 Nanoscale Flow Cytometer. Each sample was run in triplicate at a flow rate of 1.39 μL/min for a total of 2 minutes.

### Example 2: Microparticles Positive for Both PSMA and Ghrelin are Indicative of Prostate Cancer

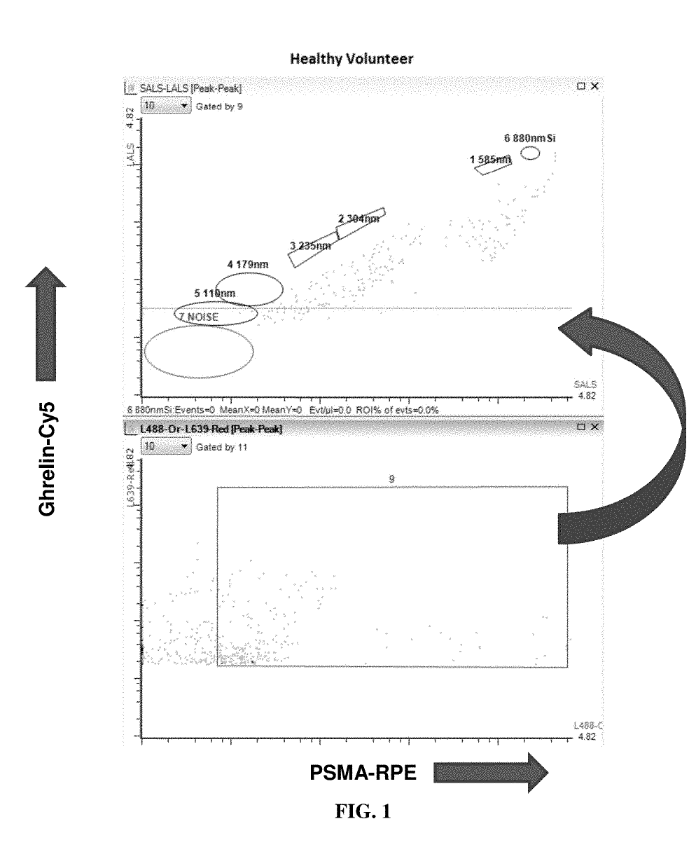

A prostate cancer microparticle (PCMP) in this assay is defined as an event that exhibits a size less than 1 μm in diameter and exhibits significant binding of both an anti-PSMA antibody pre-conjugated to a fluorophore (in this case, RPE), and Ghrelin peptide molecules (D- or L-enantiomer versions pre-conjugated to either FITC or Cy5). Incubation of patient plasma (healthy volunteer) with anti-PSMA-RPE and Ghrelin-Cy5 agents yielded a low number of dual positive events (FIG. 1, bottom panel, events within red gate). The red gate is set a priori following analysis of the same plasma sample that has been stained separately with the isotype negative controls, mouse IgG-RPE (as a negative isotypecontrol for anti-PSMA antibody) and des-acyl Ghrelin-Cy5 (wherein removal of the side chain on third amino acid prevents the peptide from binding to its receptor, GHSR). When these dual-positive events were gated onto the size histoplot (FIG. 1, top panel), resulting events exhibited a size range between 179-304 nm in diameter. These size ranges are based on the analysis of silica sizing beads that exhibit consistent size diameters (110 nm, 179 nm, 235 nm, 304 nm, 585 nm and 880 nm).

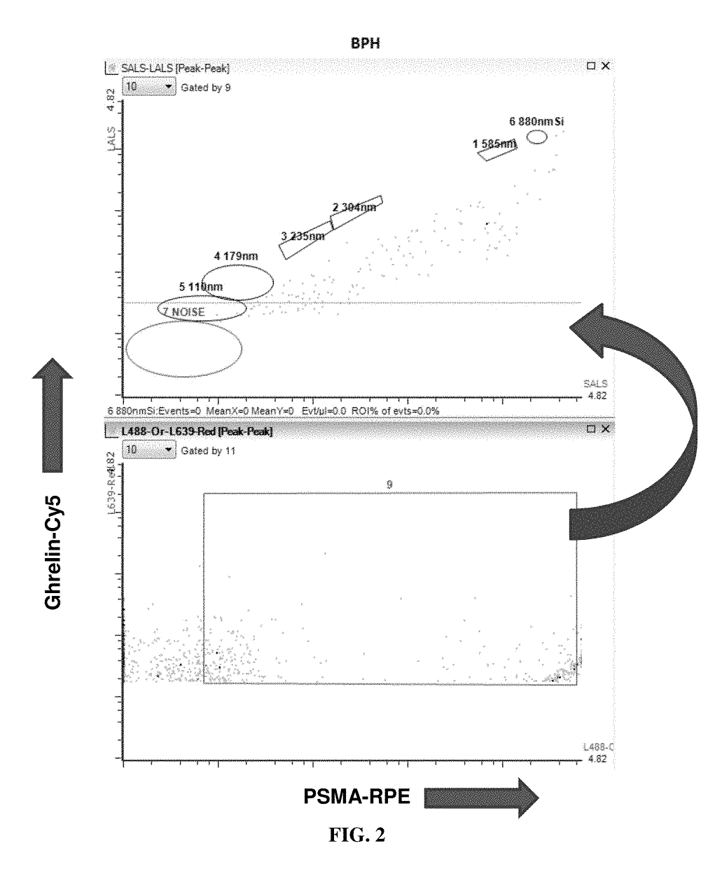

When this assay was performed on a representative plasma sample from a patient with BPH, a similar result was observed, wherein a small dual-positive subpopulation (dual-positive for anti-PSMA-RPE and Ghrelin-Cy5) was detected, as shown by events in the red gate (FIG. 2, bottom panel). When transposed onto the sizing histoplot (FIG. 2, top panel) dual-positive events had a size diameter distribution from 179 nm-304 nm, indicating that these events were indeed microparticles not background noise or soluble proteins that exhibit sizes of 0.1 nm-25 nm, which are much smaller than MPs that are 100 nm-1000 nm.

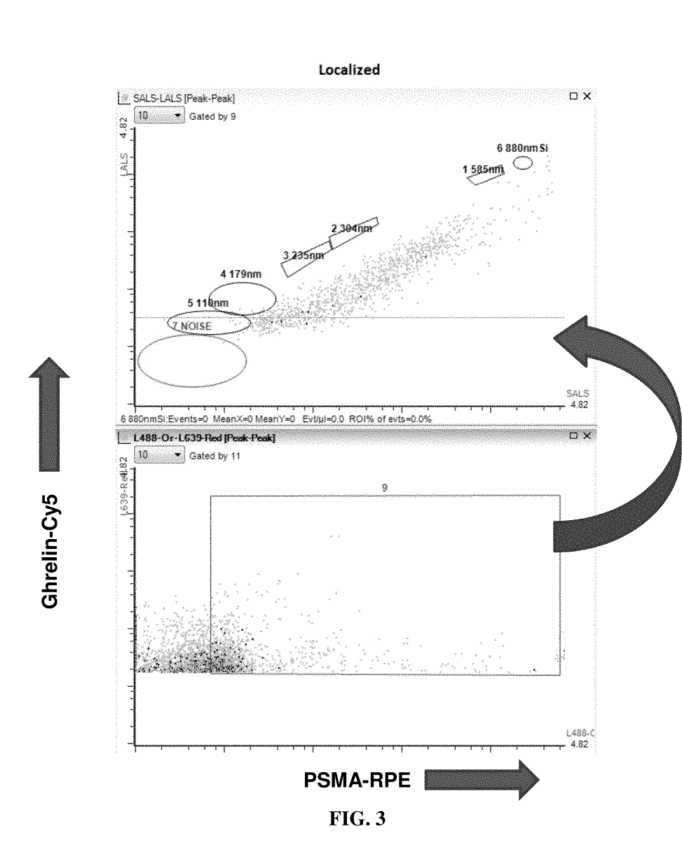

When this assay was performed on a representative plasma sample from a patient with localized prostate cancer (Gleason 7, PSA>4 ng/mL, N0, M0), a more abundant dual-positive subpopulation was observed in the red gate (FIG. 3, bottom panel). A much larger number of dual-positive events was observed relative to healthy BPH samples and, when transposed (FIG. 3, top panel) prostate cancer MP size was in a range from 179 nm-304 nm.

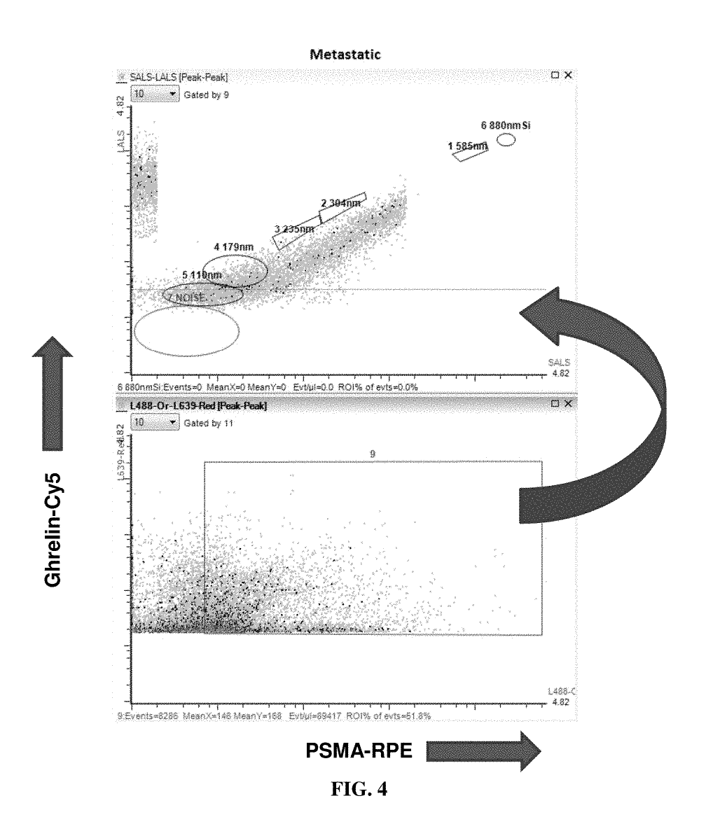

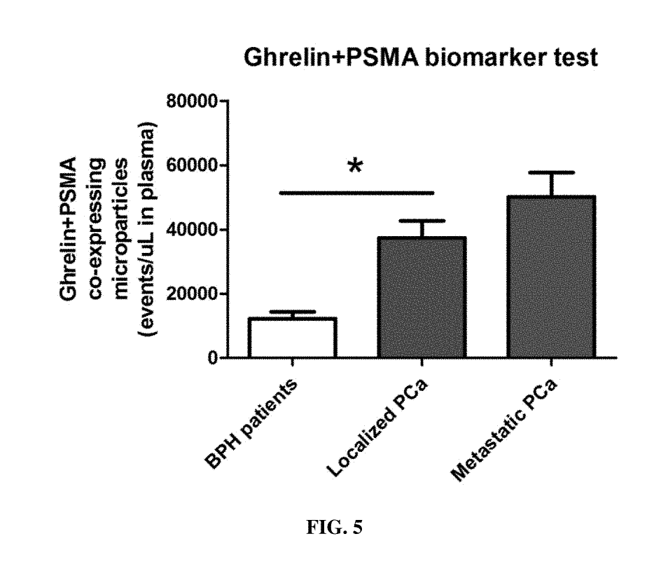

When this assay was performed on a representative plasma sample from a patient with metastatic prostate cancer (evidenced by biochemical failure, PSA>2, PSA nadir ≦0.2 ng/mL and bone scan positive for bone metastases), a dense population of dual-positive events was observed in the red gate (FIG. 4, bottom panel). When transposed onto the sizing histoplot (FIG. 5, upper panel), a size range from 110 nm-585 nm was observed, suggesting that these dual-positive events are MPs and not soluble proteins or background noise.

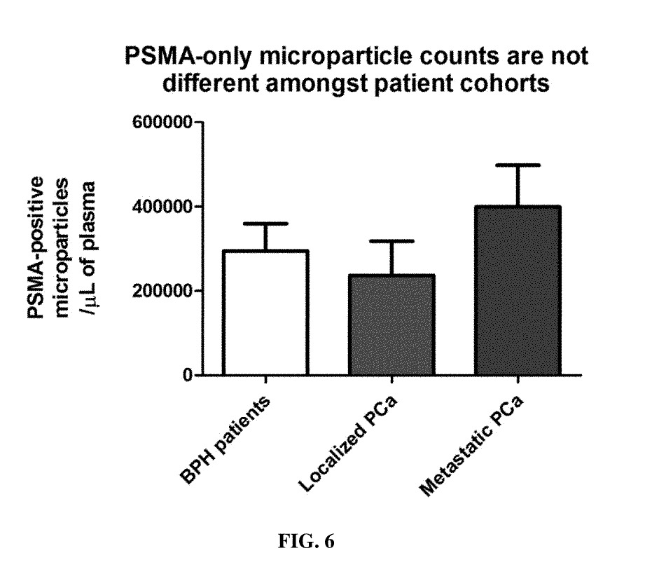

Plasmas representing patients with BPH, localized PCa and metastatic PCa were analyzed in a blinded and randomized fashion for PCMP counts (dual-positive PSMA-RPE and Ghrelin-Cy5 events). The difference in PCMP counts between the three cohorts, was largest and most statistically significant between the BPH group and the localized/metastatic PCa groups (FIG. 5). A significantly higher count was observed between BPH patients and Localized PCa or Metastatic PCa patients (*P<0.01, N>20 each group, ANOVA, bon ferroni's test). There was no statistically significant difference between the Localized PCa and Metastatic PCa groups. In this experiment, a cut-off of 17,000 PCMP counts/μL was used to distinguish patients with BPH from patients with PCa. When counts of prostate microparticles (PSMA-RPE only) were evaluated, no major differences between the groups were observed (FIG. 6). There was no statistically significant difference between any of the groups in terms of prostate microparticle counts present in plasma. Therefore, detection of PCMPs, defined as binding with both PSMA-RPE IgG and the Ghrelin-Cy5 peptide, was the only quantifiable parameter that enabled a distinction between BPH and PCa patient samples.

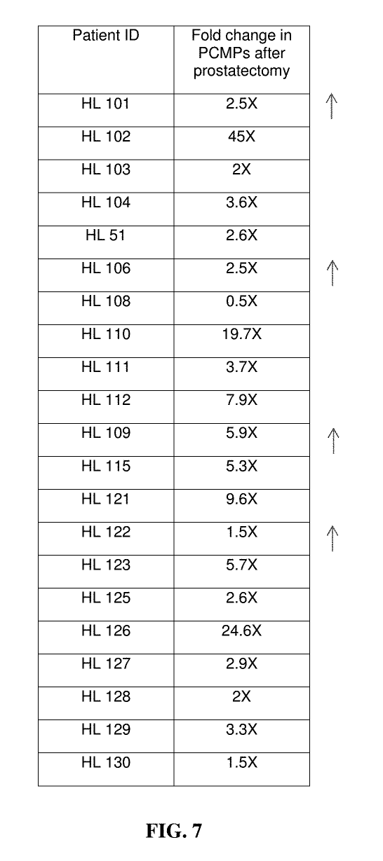

To monitor post-prostatectomy patient outcome (surgical removal of prostate and the tumor), blood was collected from patients before surgery and 3-weeks after prostatectomy. Upon analysis of plasmas from these serially collected whole bloods, it was found that a subpopulation of patients exhibited a fold increase in PCMP counts whereas the majority of patients exhibited a fold decrease in PCMP counts (FIG. 7).

Although the invention has been described with reference to certain specific embodiments, various modifications thereof will be apparent to those skilled in the art without departing from the purpose and scope of the invention as outlined in the claims appended hereto.

Any examples provided herein are included solely for the purpose of illustrating the invention and are not intended to limit the invention in any way. Any drawings provided herein are solely for the purpose of illustrating various aspects of the invention and are not intended to be drawn to scale or to limit the invention in any way. The disclosures of all prior art recited herein are incorporated herein by reference in their entirety.

We claim:

1. A method for diagnosing prostate cancer in a subject, the method comprising:
a) analyzing a bodily fluid sample to detect in the bodily fluid microparticles having at least first and second biomarkers on their surface, wherein the first biomarker is expressed by prostate epithelial cells and the second biomarker is expressed by prostate cancer cells but not by benign prostatic hyperplasic cells or other non-malignant prostate cells;
b) comparing the amount of microparticles positive for both the first and second biomarkers with a reference value,
wherein if the reference value is derived from a non-malignant prostatic sample then a detected value above the reference value is indicative of prostate cancer and a detected value equal to or below the reference value is indicative of a non-malignant disease state and,
wherein if the reference value is derived from a malignant prostatic sample then a detected value equal to or above the reference value is indicative of prostate cancer and a detected value below the reference value is indicative of a non-malignant disease state; and
c) diagnosing the subject on the basis of the results obtained in step (b).

2. The method of claim 1, wherein the bodily fluid is blood.

3. The method of claim 1 or 2, wherein the first biomarker is prostate-specific membrane antigen (PSMA), prostate stem cell antigen (PSCA), STEAP1 or STEAP2 and wherein the second biomarker is Ghrelin or C35.

4. The method of claim 3, wherein the first biomarker is PSMA.

5. The method of any one of claims 1 to 4, wherein the second biomarker is Ghrelin.

6. The method of any one of claims 1 to 5 further comprising:
d) effectuating a treatment based on the diagnosis of step (c).

7. The method of any one of claims 1 to 6, wherein the analysis of bodily fluid is conducted using a flow cytometry assay.

8. The method of claim 7, and wherein said flow cytometry assay comprises exposing the bodily fluid sample to a composition, the composition comprising a first labeled binding probe that is specific to the first biomarker and a second labeled binding probe that is specific to the second biomarker, wherein the labels of the first and second probes are distinguishable.

9. The method of claim 7 or 8, wherein the flow cytometry assay is carried out using a nanoscale flow cytometer.

10. The method of claim 8 or 9, wherein the first labeled binding probe is anti-PSMA-RPE IgG.

11. The method of any one of claims 8 to 10, wherein the second labeled probe is Ghrelin-Cy5 or Ghrelin-FITC.

12. The method of any one of claims 1 to 11, wherein the analysis is carried out with reference to at least one negative control of the first and second binding probes, wherein the at least one negative control comprises first and second negative control binding probes.

13. The method of claim 12, wherein the first negative control binding probe is mouse IgG-RPE.

14. The method of claim 12 or 13, wherein the second negative control binding probe is des-acyl Ghrelin-Cy5 or des-acyl Ghrelin-FITC.

15. The method of any one of claims 1 to 14, wherein the reference value represents the amount of microparticles positive for the first and second biomarkers in a sample obtained from a subject having a non-malignant prostate or benign prostatic hyperplasia (BPH) and wherein the detected value is an increase relative to the reference value.

16. The method of claim 15, wherein the reference value is in a range of 14,000 to 21,000 prostate cancer microparticle (PCMP) counts/μL.

17. The method of claim 16, wherein the reference value is about 17,000 PCMP counts/μL.

18. A diagnostic assay for prostate cancer, the assay comprising:
a) analyzing a bodily fluid sample to detect microparticles having at least first and second biomarkers on their surface in the bodily fluid sample, wherein the first biomarker is expressed by prostate epithelial cells and the second biomarker is expressed by prostate cancer (PCa) cells but not by benign prostatic hyperplasia or other non-malignant prostate cells;
b) comparing the amount of microparticles positive for both the first and second biomarkers with a reference value,
wherein if the reference value is derived from a non-malignant prostatic sample then a detected value above the reference value is indicative of prostate cancer and a detected value equal to or below the reference value is indicative of a non-malignant disease state and,
wherein if the reference value is derived from a malignant prostatic sample then a detected value equal to or above the reference value is indicative of prostate cancer and a detected value below the reference value is indicative of a non-malignant disease state; and
c) diagnosing the subject on the basis of the results obtained in step (b).

19. The method of claim 18, wherein the bodily fluid is blood.

20. The method of claim 18 or 19, wherein the first biomarker is prostate-specific membrane antigen (PSMA), prostate stem cell antigen (PSCA), STEAP1 or STEAP2 and wherein the second biomarker is Ghrelin or C35.

21. The method of claim 20, wherein the first biomarker is PSMA.

22. The method of any one of claims 18 to 21, wherein the second biomarker is Ghrelin.

23. The method of any one of claims 18 to 22 further comprising:
d) effectuating a treatment based on the diagnosis of step (c).

24. The method of any one of claims 18 to 23, wherein the analysis of bodily fluid is conducted using a flow cytometry assay.

25. The method of claim 24, and wherein said flow cytometry assay comprises exposing the bodily fluid sample to a composition, the composition comprising a first labeled binding probe that is specific to the first biomarker and a second labeled binding probe that is specific to the second biomarker, wherein the labels of the first and second probes are distinguishable.

26. The method of claim 24 or 25, wherein the flow cytometry assay is carried out using a nanoscale flow cytometer.

27. The method of claim 25 or 26, wherein the first labeled binding probe is anti-PSMA-RPE IgG.

28. The method of any one of claims 25 to 27, wherein the second labeled probe is Ghrelin-Cy5 or Ghrelin-FITC.

29. The method of any one of claims 18 to 28, wherein the analysis is carried out with reference to at least one negative control of the first and second binding probes, wherein the at least one negative control comprises first and second negative control binding probes.

30. The method of claim 29, wherein the first negative control binding probe is mouse IgG-RPE.

31. The method of claim 29 or 30, wherein the second negative control binding probe is des-acyl Ghrelin-Cy5 or des-acyl Ghrelin-FITC.

32. The method of any one of claims 18 to 31, wherein the reference value represents the amount of microparticles positive for the first and second biomarkers in a sample obtained from a subject having a non-malignant prostate or benign prostatic hyperplasia (BPH) and wherein the detected value is an increase relative to the reference value.

33. The method of claim 32, wherein the reference value is in a range of 14,000 to 21,000 prostate cancer microparticle (PCMP) counts/μL.

34. The method of claim 33, wherein the reference value is about 17,000 PCMP counts/μL.

35. A method for monitoring prostate cancer in a subject, the method comprising:
a) analyzing a first bodily fluid sample, wherein the first sample was obtained from the subject at a first time point, to detect microparticles having at least first and second biomarkers on their surface in the bodily fluid sample, wherein the first biomarker is expressed in prostate epithelial cells and the second biomarker is expressed by prostate cancer (PCa) cells but not by benign prostatic hyperplasia or other non-malignant prostate cells;
b) comparing the amount of microparticles positive for both the first and second biomarkers with a reference value,
wherein if the reference value is derived from a non-malignant prostatic sample then a detected value above the reference value is indicative of prostate cancer and a detected value equal to or below the reference value is indicative of a non-malignant disease state and,
wherein if the reference value is derived from a malignant prostatic sample then a detected value equal to or above the reference value is indicative of prostate cancer and a detected value below the reference value is indicative of a non-malignant disease state;
c) diagnosing the subject on the basis of the results obtained in step (b);
d) effectuating a treatment regimen based diagnosis obtained in step (c);
e) analyzing a second bodily fluid sample, wherein the second sample was obtained from the subject at a second time point, to detect microparticles having at least first and second biomarkers on their surface in the bodily fluid sample, wherein the first biomarker is expressed in prostate epithelial cells and the second biomarker is expressed by prostate cancer (PCa) cells but not by benign prostatic hyperplasia or other non-malignant prostate cells;
f) comparing the amount of microparticles positive for both the first and second biomarkers with a reference value,
wherein if the reference value is derived from a non-malignant prostatic sample then a detected value above the reference value is indicative of prostate cancer and a detected value equal to or below the reference value is indicative of a non-malignant disease state and,
wherein if the reference value is derived from a malignant prostatic sample then a detected value equal to or above the reference value is indicative of prostate cancer and a detected value below the reference value is indicative of a non-malignant disease state;
g) comparing the amount of microparticles positive for the first and second biomarker in the second bodily fluid sample with a the value obtained in step (b) wherein an increase in the amount of microparticles positive for the first and second biomarkers relative to the value obtained in step (b) is indicative of a worsened disease state and a decrease in the amount of microparticles positive for the first and second biomarkers relative to the value obtained in step (b) is indicative of an improved disease state; and
h) diagnosing any change in the subject's disease state on the basis of the results obtained in step (g).

36. The method of claim 35, wherein the bodily fluid is blood.

37. The method of claim 35 or 36, wherein the first biomarker is prostate-specific membrane antigen (PSMA), prostate stem cell antigen (PSCA), STEAP1 or STEAP2 and wherein the second biomarker is Ghrelin or C35.

38. The method of claim 37, wherein the first biomarker is PSMA.

39. The method of any one of claims 35 to 38, wherein the second biomarker is Ghrelin.

40. The method of any one of claims 35 to 39 further comprising:
d) effectuating a treatment based on the diagnosis of step (c).

41. The method of any one of claims 35 to 40, wherein the analysis of bodily fluid is conducted using a flow cytometry assay.

42. The method of claim 41, and wherein said flow cytometry assay comprises exposing the bodily fluid sample to a composition, the composition comprising a first labeled binding probe that is specific to the first biomarker and a second labeled binding probe that is specific to the second biomarker, wherein the labels of the first and second probes are distinguishable.

43. The method of claim 41 or 42, wherein the flow cytometry assay is carried out using a nanoscale flow cytometer.

44. The method of claim 42 or 43, wherein the first labeled binding probe is anti-PSMA-RPE IgG.

45. The method of any one of claims 42 to 44, wherein the second labeled probe is Ghrelin-Cy5 or Ghrelin-FITC.

46. The method of any one of claims 35 to 45, wherein the analysis is carried out with reference to at least one negative control of the first and second binding probes, wherein the at least one negative control comprises first and second negative control binding probes.

47. The method of claim 46, wherein the first negative control binding probe is mouse IgG-RPE.

48. The method of claim 46 or 47, wherein the second negative control binding probe is des-acyl Ghrelin-Cy5 or des-acyl Ghrelin-FITC.

49. The method of any one of claims 35 to 48, wherein the reference value represents the amount of microparticles positive for the first and second biomarkers in a sample obtained from a subject having a non-malignant prostate or benign prostatic hyperplasia (BPH) and wherein the detected value is an increase relative to the reference value.

50. The method of claim 49, wherein the reference value is in a range of 14,000 to 21,000 prostate cancer microparticle (PCMP) counts/μL.

51. The method of claim 50, wherein the reference value is about 17,000 PCMP counts/μL.

52. A method for assessing efficacy of a therapy on a subject having prostate cancer, the method comprising:
a) analyzing a bodily fluid sample from a subject, wherein the subject has be subjected to a prostate cancer therapy, to detect microparticles having at least first and second biomarkers on their surface in the bodily fluid sample, wherein the first biomarker is expressed in prostate cells and the second biomarker is expressed by prostate cancer (PCa) cells but not by benign prostatic hyperplasia or other non-malignant prostate cells;
b) comparing the amount of microparticles positive for both the first and second biomarkers with a reference value,
wherein if the reference value is derived from a non-malignant prostatic sample then a detected value above the reference value is indicative of prostate cancer and a detected value equal to or below the reference value is indicative of a non-malignant disease state and,
wherein if the reference value is derived from a malignant prostatic sample then a detected value equal to or above the reference value is indicative of prostate cancer and a detected value below the reference value is indicative of a non-malignant disease state; and
c) diagnosing the efficacy of the therapy as good if the value obtained in step (b) indicates a non-malignant disease state or poor if the value obtained in step (b) indicates prostate cancer.

53. The method of claim 52, wherein the bodily fluid is blood.

54. The method of claim 52 or 53, wherein the first biomarker is prostate-specific membrane antigen (PSMA), prostate stem cell antigen (PSCA), STEAP1 or STEAP2 and wherein the second biomarker is Ghrelin or C35.

55. The method of claim 54, wherein the first biomarker is PSMA.

56. The method of any one of claims 52 to 55, wherein the second biomarker is Ghrelin.

57. The method of any one of claims 52 to 56 further comprising:
d) effectuating a treatment based on the diagnosis of step (c).

58. The method of any one of claims 52 to 57, wherein the analysis of bodily fluid is conducted using a flow cytometry assay.

59. The method of claim 58, and wherein said flow cytometry assay comprises exposing the bodily fluid sample to a composition, the composition comprising a first labeled binding probe that is specific to the first biomarker and a second labeled binding probe that is specific to the second biomarker, wherein the labels of the first and second probes are distinguishable.

60. The method of claim 58 or 59, wherein the flow cytometry assay is carried out using a nanoscale flow cytometer.

61. The method of claim 59 or 60, wherein the first labeled binding probe is anti-PSMA-RPE IgG.

62. The method of any one of claims 59 to 61, wherein the second labeled probe is Ghrelin-Cy5 or Ghrelin-FITC.

63. The method of any one of claims 52 to 62, wherein the analysis is carried out with reference to at least one negative control of the first and second binding probes, wherein the at least one negative control comprises first and second negative control binding probes.

64. The method of claim 63, wherein the first negative control binding probe is mouse IgG-RPE.

65. The method of claim 63 or 64, wherein the second negative control binding probe is des-acyl Ghrelin-Cy5 or des-acyl Ghrelin-FITC.

66. The method of any one of claims 52 to 65, wherein the reference value represents the amount of microparticles positive for the first and second biomarkers in a sample obtained from a subject having a non-malignant prostate or benign prostatic hyperplasia (BPH) and wherein the detected value is an increase relative to the reference value.

67. The method of claim 66, wherein the reference value is in a range of 14,000 to 21,000 prostate cancer microparticle (PCMP) counts/μL.

68. The method of claim 67, wherein the reference value is about 17,000 PCMP counts/μL.

69. A method of any one of claims 1 to 68, wherein the subject is a mammal.

70. The method of claim 69, wherein the mammal is a human.

71. A kit for detecting prostate cancer in a bodily fluid sample, the kit comprising
a first binding probe specific to a first biomarker that is expressed by prostate epithelial cells, and
a second binding probe specific to a second biomarker that is expressed by prostate cancer (PCa) cells but not by benign prostatic hyperplasia or other non-malignant prostate cells.

72. The kit of claim 71, wherein the first biomarker is prostate-specific membrane antigen (PSMA), prostate stem cell antigen (PSCA), STEAP1 or STEAP2 and wherein the second biomarker is Ghrelin or C35.

73. The kit of claim 72, wherein the first biomarker is PSMA.

74. The kit of claim 72 or 73, wherein the first binding probe is anti-PSMA-RPE IgG.

75. The kit of any one of claims 71 to 74, wherein the second biomarker is Ghrelin.

76. The kit of any one of claims 71 to 75, wherein the second binding probe is Ghrelin-Cy5 or Ghrelin-FITC.

77. The kit of any one of claims 71 to 76, further comprising a first negative control binding probe specific to mouse IgG.

78. The kit of claim 77, further comprising a second negative control binding probe specific to des-acyl Ghrelin.

79. The kit of claim 77 or 78, wherein the first negative control binding probe is monoclonal antibody mouse IgG-RPE.

80. The kit of claim 78 or 79, wherein the second negative control binding probe is des-acyl Ghrelin-Cy5 or des-acyl Ghrelin-FITC.

81. The kit of any one of claims 71 to 80, further comprising a first and second sealed container, wherein the first sealed container comprises anti-PSMA-RPE IgG and Ghrelin-Cy5 or Ghrelin-FITC and the second sealed container comprises mouse IgG-RPE and des-acyl Ghrelin-Cy5 or des-acyl Ghrehn-FITC.

82. The kit of any one of claims 71 to 81, further comprising a carrier, wherein a carrier is a box, carton, or tube.

83. The kit of claim 82, wherein the carrier comprises one or more sealed containers, wherein the one or more sealed container is a vial, tube, ampoule, bottle, pouch or envelope.

84. The kit of any one of claims 71 to 83, further comprising one or more media, media ingredients or reagents for measurement of at least one of the first and second biomarkers.

85. The kit of claim 84, wherein the one or more reagents are buffers or probes.

86. The kit of any one of claims 71 to 85, further comprising one or more instructions or protocols for carrying out the methods of any one of claims 1 to 70.


SEQUENCE LISTING (5 sequences)

SEQ ID NO: 1 - protein, 18 aa, Homo sapiens
  MISC_FEATURE (2)..(2): octanoyl
GSFLSPEHRQVQQRKESK

SEQ ID NO: 2 - protein, 19 aa, Artificial Sequence
  artificial binding probe
GSXFLSPEHRQVQQRKESK

SEQ ID NO: 3 - protein, 19 aa, Artificial Sequence
  artificial binding probe
GSSFLSPEHRQVQQRKESK

SEQ ID NO: 4 - protein, 19 aa, Artificial Sequence
  artificial binding probe
GSXFLSPEHRQVQQRKESK

SEQ ID NO: 5 - protein, 19 aa, Artificial Sequence
  artificial binding probe
GSSFLSPEHRQVQQRKESK